(19) United States
(12) Patent Application Publication
(10) Pub. No.: US 2016/0069830 A1
(43) Pub. Date: Mar. 10, 2016

(54) CONDUCTIVITY MEASUREMENT METHOD THAT SLOWS DOWN CONDUCTOR OXIDATION

(71) Applicant: SOLTEAM OPTO, INC., Taoyuan City (TW)

(72) Inventors: Chun-Yen WU, Taoyuan City (TW); Hsiang Chiang LIU, Taoyuan City (TW); Shang-Jung WU, Taoyuan City (TW); Shih-Hua CHEN, Taoyuan City (TW); Chin-Feng CHEN, Taoyuan City (TW)

(21) Appl. No.: 14/478,997

(22) Filed: Sep. 5, 2014

(51) Int. Cl. G01N 27/02; G01N 33/18

(52) U.S. Cl. CPC G01N 27/02; G01N 33/1886

(57) ABSTRACT

A conductivity measurement method for measuring the conductivity of a fluid in a washing chamber in an electrical washing machine using a water quality sensor consisting of a control unit and first and second conductors by alternatively changing the electric potential of the first and second conductors and repeatedly measuring the conductivity of the fluid in the washing chamber between the first and second conductors upon each electric potential alternation.

### BACKGROUND OF THE INVENTION

1. Field of the Invention

The present invention relates to conductivity measurement technology and more particularly, to a conductivity measurement method that slows down conductor oxidation by alternatively changing the electric potential of the first and second conductors of the water quality sensor during its operation to measure the conductivity of a fluid in a washing chamber of a washing machine.

2. Description of the Related Art

With the rapid progress in modern technology, many advanced electrical home appliances have been continuously created for handling labor-consuming jobs, bringing both convenience and comfort to people. Among commercial electrical home appliances, washing machines such as clothes washers or dishwashers are adapted for washing objects with water and a cleaning agent, saving much washing labor and time. A clothes washer, dishwasher or any other washing machine generally has a water quality sensor installed in the internal washing chamber for detecting the quality of the fluid in the washing chamber. A water quality sensor for this purpose generally comprises a control unit, and two conductors electrically coupled in parallel to the control unit and suspending in the washing chamber for direct contact with the applied fluid. In operation, the control unit drives one conductor into HIGH and the other into LOW, enabling an electric current to be discharged out of the conductor at HIGH and conducted by the fluid in the washing chamber to the other conductor at LOW, and thus the control unit can measure the conductivity of the fluid through the two conductors. Normally, the conductivity of electrons in the fluid is constant. If ions (laundry detergent or washing powder) exist in the fluid, the conductivity of the electrons in the fluid will be changed. By means of comparing the conductivity of the fluid in the washing chamber with a predetermined reference value can check the presence of unnecessary ions in the fluid to determine whether or not to keep washing.

However, when the control unit conducts the two conductors to measure the conductivity of the fluid, the applied electric current goes from one conductor through the fluid to the other conductor in one single direction, thus, conductor polarization can lead to oxidation of the conductors. Due to the effect of reduction at the conductor at LOW, the other conductor at HIGH can get rust when conducted for a certain length of time, affecting its current output strength and resulting in a conductivity measuring error and further washing determination error. An improvement in this regard is necessary.

### SUMMARY OF THE INVENTION

The present invention has been accomplished under the circumstances in view. It is therefore the main object of the present invention to provide a conductivity measurement method that slows down oxidation of the conductors of the water quality sensor when measuring the conductivity of a fluid in a washing chamber of a washing machine, assuring a high level of sensing accuracy and enabling the washing machine to accurately determine continuation of the washing process subject to the fluid state in the washing chamber. It is another object of the present invention to provide a conductivity measurement method that slows down oxidation of the conductors of the water quality sensor during the conductivity measuring operation, thereby prolonging the lifespan of the water quality sensor.

To achieve these and other objects of the present invention, a conductivity measurement method for measuring the conductivity of a fluid in a washing chamber in an electrical washing machine using a water quality sensor consisting of a control unit and first and second conductors is performed by alternatively changing the electric potential of the first and second conductors and repeatedly measuring the conductivity of the fluid in the washing chamber between the first and second conductors upon each electric potential alternation.

Other advantages and features of the present invention will be fully understood by reference to the following specification in conjunction with the accompanying drawings, in which like reference signs denote like components of structure.

### BRIEF DESCRIPTION OF THE DRAWINGS

FIG. 1 is an oblique top elevational view of a water quality sensor in accordance with the present invention.

FIG. 2 is a block diagram of the water quality sensor shown in FIG. 1.

FIG. 3 is a flow chart of a conductivity measurement method in accordance with the present invention.

### DETAILED DESCRIPTION OF THE PREFERRED EMBODIMENT

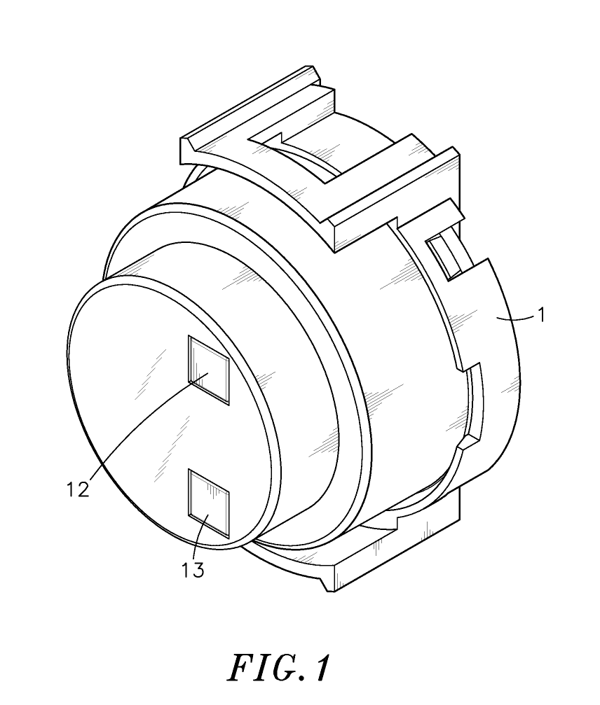

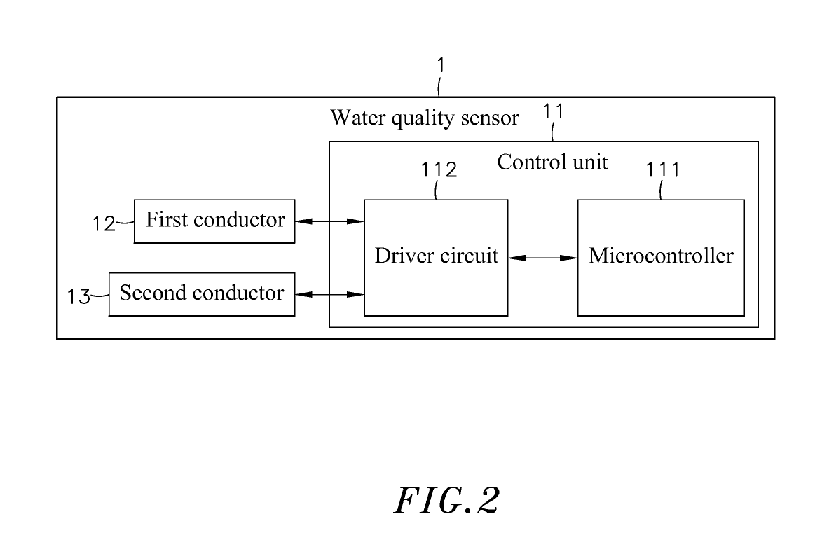

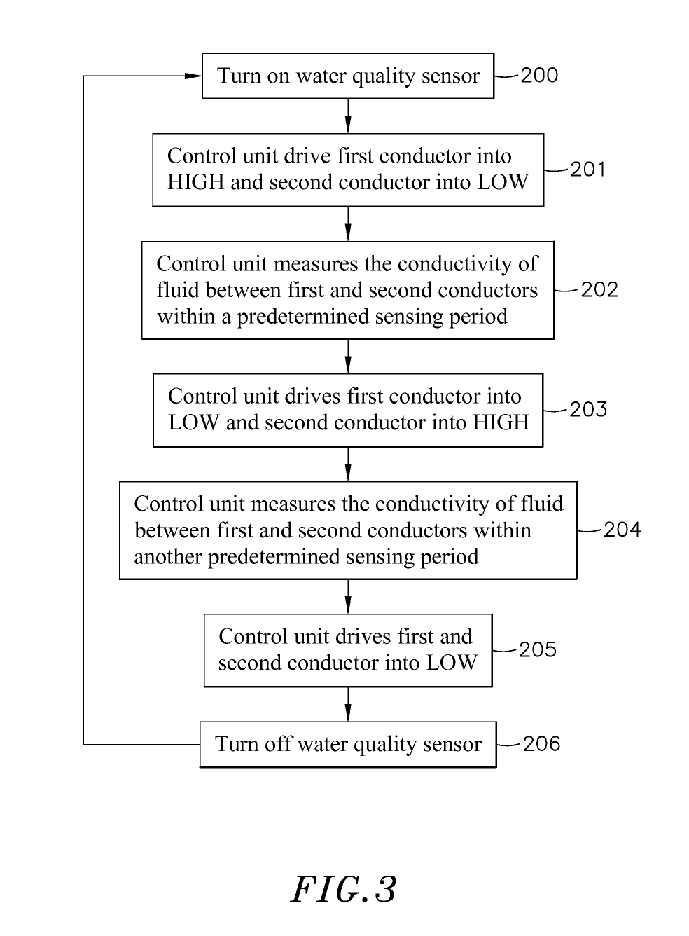

Referring to FIGS. 1 and 2, a water quality sensor 1 is shown, comprising a control unit 11, and a first conductor 12 and a second conductor 13 respectively electrically connected to the control unit 11 and suspending outside the water quality sensor 1. The control unit 11 comprises a microcontroller 111 and a driver circuit 112. The microcontroller 111 is electrically coupled with the driver circuit 112. The driver circuit 112 is electrically coupled with the first conductor 12 and the second conductor 13. Referring to FIG. 3, the water quality sensor 1 is used to perform a conductivity measurement method for measuring the conductivity of a fluid. The conductivity measurement method comprises the steps of:
(200) turning on the water quality sensor 1;
(201) the control unit 11 driving the first conductor 12 into HIGH and the second conductor 13 into LOW;
(202) the control unit 11 measuring the conductivity of the fluid between the first conductor 12 and the second conductor 13 within a predetermined sensing period;
(203) the control unit 11 driving the first conductor 12 into LOW and the second conductor 13 into HIGH;
(204) the control unit 11 measuring the conductivity of the fluid between the first conductor 12 and the second conductor 13 within another predetermined sensing period;
(205) the control unit 11 driving the first conductor 12 and the second conductor 13 into LOW;
(206) turning off the water quality sensor 1, and then returning to step (200) a predetermined sensing period after the water quality sensor 1 is off.

More specifically, in the aforesaid process, the water quality sensor 1 is installed in an electrical washing machine (clothes washer or dishwasher) to suspend the first conductor 12 and the second conductor 13 in the washing chamber. When the electrical washing machine is started up, the water quality sensor 1 is turned on. At this time, the microcontroller 111 of the control unit 11 provides a control signal to the driver circuit 112, causing the driver circuit 112 to drive the first conductor 12 into HIGH and the second conductor 13 into LOW for a predetermined sensing period and then to output an electric current through the first conductor 12 to the second conductor 13 via the fluid in the washing chamber. Within the aforesaid predetermined sensing period, the microcontroller 111 provides a sensing signal to the driver circuit 112 to detect the conductivity of the fluid between the first conductor 12 and the second conductor 13.

The driver circuit 112 of the control unit 11 is adapted for amplifying or filtrating the control signal provided by the microcontroller 111, or to dividing the voltage of the control signal. As the driver circuit is of the known art and not within the scope of the spirit of the present invention, no further detailed description in this regard will be necessary.

After the aforesaid predetermined sensing period is up, the microcontroller 111 of the control unit 11 will provide a control signal to the driver circuit 112, causing the driver circuit 112 to drive the first conductor 12 into LOW and the second conductor 13 into HIGH for another predetermined sensing period and then to output an electric current through the second conductor 13 to the first conductor 12 via the fluid in the washing chamber. Within this secondary predetermined sensing period, the microcontroller 111 provides a sensing signal to the driver circuit 112 to detect the conductivity of the fluid between the first conductor 12 and the second conductor 13.

After the aforesaid secondary predetermined sensing period is up, the microcontroller 111 of the control unit 11 will provide a control signal to the driver circuit 112, causing the driver circuit 112 to drive the first conductor 12 and the second conductor 13 into LOW, stopping conduction between the first conductor 12 and the second conductor 13, and then the water quality sensor 1 is turned off. After a predetermined period, which is determined subject to the washing operation of the electrical washing machine, the aforesaid operating process is repeated.

The aforesaid predetermined sensing period can be in the range of 3-30 seconds subject to the washing operation of the electrical washing machine, allowing the control unit 11 to detect the conductivity of the fluid between the first conductor 12 and the second conductor 13.

As stated above, the conductivity measurement method of the present invention is to alternatively change the electric potential of the first conductor 12 and the second conductor 13 subject to the control of the control unit 11, slowing down oxidation of the first conductor 12 and second conductor 13 at high potential due to polarization and preventing output current instability due to rust of the first conductor 12 and second conductor 13. Thus, the control unit 11 can accurately measure the conductivity of the fluid through the first conductor 12 and second conductor 13, enabling the washing machine to accurately determine continuation of the washing process subject to the fluid state in the washing chamber.

Further, controlling the control unit 11 of the water quality sensor 1 to alternatively change the electric potential of the first conductor 12 and second conductor 13 during the measuring process can effectively slows down oxidation of the first conductor 12 and second conductor 13, preventing the first conductor 12 and second conductor 13 from getting rust and prolonging the lifespan of the water quality sensor 1.

In conclusion, the conductivity measurement method of the present invention has the advantages as follows:

1. By means of the control unit 11 of the water quality sensor 1 to alternatively change the electric potential of the first conductor 12 and second conductor 13 during the measuring process, oxidation of the first conductor 12 and second conductor 13 at high potential is slowed down, achieving a high level of conductivity measuring accuracy.

2. Controlling the control unit 11 of the water quality sensor 1 to alternatively change the electric potential of the first conductor 12 and second conductor 13 during the measuring process can prevent the first conductor 12 and second conductor 13 from getting rust and prolonging the lifespan of the water quality sensor 1.

Although a particular embodiment of the invention has been described in detail for purposes of illustration, various modifications and enhancements may be made without departing from the spirit and scope of the invention. Accordingly, the invention is not to be limited except as by the appended claims.

What the invention claimed is:

1. A conductivity measurement method for measuring the conductivity of a fluid in a washing chamber in an electrical washing machine using a water quality sensor that comprises a control unit, and a first conductor and a second conductor respectively electrically coupled to said control unit and suspending in said washing chamber, the conductivity measurement method comprising the steps of:
(A01) turning on said water quality sensor;
(A02) said control unit driving said first conductor into HIGH and said second conductor into LOW;
(A03) said control unit measuring the conductivity of said fluid between said first conductor and said second conductor within a predetermined sensing period;
(A04) said control unit driving said first conductor into LOW and said second conductor into HIGH;
(A05) said control unit measuring the conductivity of said fluid between said first conductor and said second conductor within another said predetermined sensing period;
(A06) said control unit driving said first conductor and said second conductor into LOW; and
(A07) turning off said water quality sensor.

2. The conductivity measurement method as claimed in claim 1, wherein said predetermined sensing period is in the range of 3-30 seconds.

3. The conductivity measurement method as claimed in claim 1, wherein said step (A07) further comprises a sub step of returning to step (A01) a predetermined period after said water quality sensor is off.

4. The conductivity measurement method as claimed in claim 1, wherein said control unit comprises a microcontroller, and a driver circuit electrically coupled to said microcontroller; said first conductor and said second conductor are respectively electrically coupled to said driver circuit.

5. The conductivity measurement method as claimed in claim 4, wherein said microcontroller is adapted to provide a control signal to said driver circuit, causing said driver circuit to change the electric potential of said first conductor and said second conductor.

6. The conductivity measurement method as claimed in claim 4, wherein said microcontroller is adapted to provide a sensing signal to said driver circuit, causing said driver circuit to detect the conductivity in said fluid between said first conductor and said second conductor.
